(19) United States
(12) Patent Application Publication
(10) Pub. No.: US 2002/0034752 A1
(43) Pub. Date: Mar. 21, 2002

(54) CETP TaqIB polymorphism as risk factor for development of coronary heart disease

(76) Inventors: Jose M. Ordovas, Framingham, MA (US); Ernst J. Schaefer, Wellesley, MA (US)

Correspondence Address: Farrell & Associates, P.C., P.O. Box 999, York Harbor, ME, 03911, US

(21) Appl. No.: 09/852,980

(22) Filed: May 10, 2001

Related U.S. Application Data
(60) Provisional application No. 60/203,467, filed on May 11, 2000.

(51) Int. Cl. C12Q 1/68; C12P 19/34

(52) U.S. Cl. 435/6; 435/91.2

(57) ABSTRACT

Disclosed is method for assessing risk for the development of cardiovascular disease in an individual. The method includes isolating nucleic acid from the individual, analyzing the nucleic acid for the presence of the TaqIB polymorphism of the cholesteryl ester transfer protein gene, determining from the analysis whether the individual is homozygous for the TaqIB polymorphism; is heterozygous for the TaqIB polymorphism; or does not possess the TaqIB polymorphism. Risk for the development of cardiovascular disease is assessed in the individual on the basis of these determinations. Additional determinations of one or more known factors of cardiovascular disease risk may also be assessed. Methods for analysis of genomic DNA for the presence of the TaqIB polymorphism are provided. Also disclosed is a kit for assessing risk for the development of cardiovascular disease in an individual. The kit contains useful reagents, such as oligonucleotide primers for the amplification of a suitable section of the first intron of the cholesteryl ester transfer protein gene encompassing the TaqI restriction site of the B1 allele of the CETP gene. Optionally, the kit also contains indicators for additional known factors of cardiovascular disease risk.

### GOVERNMENT SUPPORT

This work was supported by grants HL54776 and NIH/NHLBI, contract NO1-38038 and contract 53-K06-5-10, from the US Department of Agriculture Research Service, and as such the U.S. Government owns certain rights in the invention.

### BACKGROUND OF THE INVENTION

Cholesteryl ester transfer protein (CETP) facilitates the exchange of triglycerides and cholesteryl esters between lipoprotein particles. In humans, CETP mRNA encodes a polypeptide of MR 53,000, which is n-glycosylated at four sites, giving rise to the mature form of CETP of MR 74,000 (Drayna et al., Nature 327: 632-634 (1987)). CETP is expressed primarily in liver, spleen and adipose tissue, and lower levels have been detected in the small intestine, adrenal gland, heart, kidney and skeletal muscle (Drayna et al., Nature 327: 632-634 (1987); Bruce and Chouinard Jr., Annu. Rev. Nutr. 18: 297-330 (1998)). The CETP gene encompasses 16 exons, and it has been localized on chromosome 16q21 adjacent to the LCAT gene. Several mutations at the CETP locus have been identified resulting in absence of detectable CETP mass and/or activity (Yamashita et al., Curr. Opin. Lipidol. 8: 101-110 (1997)). These mutations are common in Japanese populations (Inazu et al., N. Engl. J. Med. 323: 1234-1238 (1990); Koizumi et al., Atherosclerosis 90: 189-196 (1991); Takegoshi et al., Atherosclerosis 96: 83-85 (1992); Inazu et al., J. Clin. Invest. 94: 1872-1882 (1994)) although some have been recently reported in Caucasian subjects (Hill et al., Clin. Biochem. 30: 413-418 (1997); Tamminen et al., Atherosclerosis 124: 237-247 (1996)). CETP deficiency is associated with hyperalphalipoproteinemia, primarily due to an increase of cholesteryl ester-enriched large size HDL. Conversely, the triglyceride rich lipoproteins and the LDL are smaller and triglyceride enriched, reflecting its role in neutral lipid exchange (Yamashita et al., Curr. Opin. Lipidol. 8: 101-110 (1997).

Several common restriction fragment length polymorphisms (RFLPs) have been reported in the CETP gene locus (Drayna and Lawn, Nucleic Acids Res. 15: 4698 (1987); Freeman et al., Nucleic Acids Res. 17: 2880 (1989); Zuliani and Hobbs, Nucleic Acids Res. 18: 2834 (1990)). The most studied RFLP to date has been the TaqIB, which has been shown to be a silent base change affecting the 277^{th} nucleotide in the first intron of the gene (Drayna and Lawn, Nucleic Acids Res. 15: 4698 (1987)). The B2 allele (absence of the TaqI restriction site) at this polymorphic site has been associated in normolipemic subjects with increased HDL-C levels and decreased CETP activity and levels (Kondo et al., Clin. Genet. 35: 49-56 (1989); Freeman et al., Arterioscler. Thromb. 14: 336-344 (1994); Hannuksela et al., Atherosclerosis 110: 35-44 (1994); Kuivenhoven et al., Arterioscler. Thromb. Vasc. Biol. 17: 560-568 (1997)), thus, resembling a mild form of CETP deficiency. It has been suggested that this association may be population specific (Tenkanen et al., Hum. Genet. 87: 574-578 (1991); Mitchell et al., Human Biology 66: 13-25 (1994)) and highly influenced by environmental factors such as alcohol consumption and tobacco smoking (Hannuksela et al., Atherosclerosis 110: 35-44 (1994); Fumeron et al., J. Clin. Invest. 96: 1664-1671 (1995); Kauma et al., Hum. Genet. 97: 156-162 (1996)). Moreover, Kuivenhoven et al. (N. Engl. J. Med. 338: 86-93 (1998)) has shown an interaction between the TaqIB genotype and the progression of coronary heart disease following therapy. These observations could be of significant relevance, since low plasma HDL levels are associated with an increase in coronary artery disease risk (Gordon et al., Am. J. Med. 62: 707-714 (1977); Gordon and Rifkind, N. Engl. J. Med. 321: 1311-1316 (1989)). Moreover, clinical evidence suggests that an increase of 1% in the plasma HDL-C levels is associated with a reduction in cardiovascular morbidity and mortality of 2-3% (Manninen et al., JAMA 260: 641-651 (1988)). Therefore, CETP could have a relevant role in atherogenesis through its effects on HDL metabolism.

### BRIEF DESCRIPTION OF THE FIGURES

FIG. 1 is a graphical representation of data from sensitivity analysis of six different models. Regression coefficients and 95% confidence intervals for B1B2 and B2B2 genotypes, respectively, are compared with B1B1 when each indicated variable was progressively included into the linear regression models. The respective models include the following: Model 1: CETP genotype; Model 2: Model 1+gender; Model 3: Model 2+body mass index (BMI); Model 4: Model 3+tobacco smoking; Model 5: Model 4+alcohol consumption; Model 6: Model 5+ApoE genotype. R-squared were included in the figure to show the variability accounted for each regression model.

### SUMMARY OF THE INVENTION

The present invention relates to a method for assessing risk for the development of cardiovascular disease in an individual. The method comprises isolating nucleic acid from the individual, analyzing the nucleic acid for the presence of the TaqIB polymorphism of the cholesteryl ester transfer protein gene, determining from the analysis whether the individual is homozygous for the TaqIB polymorphism; is heterozygous for the TaqIB polymorphism; or does not possess the TaqIB polymorphism. Risk for the development of cardiovascular disease is assessed in the individual on the basis of these determinations. In one embodiment, additional determinations of one or more known factors of cardiovascular disease risk are also assessed. In a preferred embodiment, the genomic DNA is analyzed for the presence of the TaqIB polymorphism by restriction analysis of an amplified fragment for the presence of a TaqI restriction site at a position corresponding to nucleotide 277 of the first intron. Useful primers for PCR amplification of a suitable fragment are provided.

Another aspect of the present invention relates to a kit for assessing risk for the development of cardiovascular disease in an individual. The kit comprises oligonucleotide primers for the amplification of a suitable section of the first intron of the cholesteryl ester transfer protein gene encompassing the TaqI restriction site of the B1 allele of the CETP gene. The kit optionally further comprises indicators for additional known factors of cardiovascular disease risk.

### DETAILED DESCRIPTION OF THE INVENTION

Cholesteryl ester transfer protein (CETP) facilitates the exchange of triglycerides and cholesteryl esters between lipoprotein particles, a key step in reverse cholesterol transport in humans. Variations at the CETP locus have previously been shown to be determinants of the levels and activity of CETP and high density lipoprotein plasma concentration. One common variation of the CETP locus is the CETP gene polymorphism, TaqIB (referred to herein as the TaqIB polymorphism) which is located in intron 1. The present invention is based on the identification of a statistically significant correlation of the absence of the TaqIB polymorphism with the frequency, phenotypic expression and potential modulation of coronary heart disease (also referred to herein as cardiovascular disease) development in the general population.

Detailed in the Exemplification section below is an analysis of the association of the TaqIB polymorphism with interindividual variability in lipid levels, lipoprotein subclass profiles, CETP activity, and cardiovascular disease risk, examined in a population-based sample of 1411 men and 1505 women from the Framingham Offspring Study. The findings reveal a correlation of the absence of the TaqIB polymorphism (denoted as the homozygous presence of the B1 allele) with the development of cardiovascular disease/coronary heart disease. Absence of the TaqIB polymorphism (presence of the B1 allele) also correlates with decreased HDL-C levels in men and women, and also with decreased apoA-I levels in men. The presence of the TaqIB polymorphism (denoted as presence of the B2 allele) correlates with about 30% lower risk of developing coronary heart disease.

These findings are directly applicable to methods for ascertaining predisposition to disease development. One aspect of the present invention relates to a method for assessing risk for the development of cardiovascular disease in an individual by examination of the individual for the presence or absence of the TaqIB polymorphism. The term cardiovascular disease as used herein includes, without limitation, conditions such as coronary artery disease, myocardial infarction, angina pectoris, coronary insufficiency and coronary death.

The method involves isolation of nucleic acid from an individual, followed by analysis of the nucleic acid for the presence or absence of the TaqIB polymorphism. This analysis is used to determine if the individual is homozygous for the TaqIB polymorphism (B2B2), is heterozygous for the TaqIB polymorphism (B1B2), or does not possess the TaqIB polymorphism (B1B1). Once the genotype of the individual is determined, the risk for the development of cardiovascular disease in the individual is assessed on the basis of this genotype determination utilizing the correlations presented in the Exemplification section below.

To determine risk of disease development in an individual, one applies statistically significant correlations made in a population between disease development and presence of a given factor or factors, to the individual. Risk, as the term is used herein, refers to the likelihood of disease development. Risk is determined by consideration of one or more disease factors present in, or associated with, the individual. A factor, or risk factor, is a specific condition of an individual (e.g., genotype, physiologic state, behavior, and environmental condition) which has a documented, statistically significant correlation with development of the disease in question. The factor may be known to contribute to disease progression or merely known associated with disease development. Risk is generally used to describe an increased likelihood of disease development, but may also describe a decreased likelihood (e.g., protection). A determination of decreased likelihood, generally referred to as decreased risk, is often made with respect to consideration of other known (increased) risk factors. As an application of statistical analysis to real life predispositions, risk is conceptually determined relative to an otherwise similar individual having a different complement of all factors being considered (e.g., genetic or behavioral/environmental).

The TaqIB polymorphism exhibits codominance for the observed phenotypes. A determination that the individual does not possess the TaqIB polymorphism indicates a high increased risk for the development of cardiovascular disease, relative to a determination that the individual is homozygous for the TaqIB polymorphism. A determination that the individual is heterozygous for the TaqIB polymorphism indicates a moderate increased risk for cardiovascular disease development relative to a determination that the individual is homozygous for the TaqIB polymorphism. A determination that the individual is homozygous for the TaqIB polymorphism indicates no increased risk for the cardiovascular disease development. Indeed, such a determination may actually indicate decreased risk in the form of protection from the disease when considered with other known factors of cardiovascular disease development.

Preferably, the risk for the development of cardiovascular disease in the individual is assessed on the basis of the presence or absence of the TaqIB polymorphism in combination with additional determinations of one or more known factors of cardiovascular disease risk. Because the development of cardiovascular disease is influenced by a variety of factors, both genetic and environmental, the risk for disease development is optimally determined by consideration of as many factors as possible. Other known genetic factors include, without limitation, apolipoprotein E, lipoprotein lipase, and the low density lipoprotein (LDL) receptor of the individual. Mutations in the individual's angiotensin-converting enzyme gene have also been identified as factors in the development of cardiovascular disease. Specific mutations and methods for their identification is disclosed in Raynolds et al., U.S. Pat. No. 5,800,990 (1998), the contents of which are incorporated herein by reference. Environmental factors include, without limitation, diet (e.g., fat and cholesterol), level of exercise, alcohol consumption, and smoking. Each of these factors contributes to the susceptibility or protection of the individual from cardiovascular disease. Therefore, the overall risk of the individual is best assessed by taking as many known factors into account as possible.

In addition, several physiologic factors (caused by either genetic or environmental factors) also play a significant role in the development of cardiovascular disease. Examples of such are age, weight, blood pressure (systolic and diastolic), lipid parameters (e.g., total cholesterol, triglycerides, low and high density lipoproteins), and glycemic parameters (glucose and/or insulin). Elevated plasma homocysteine levels are also used to indicate substantially increased risk of coronary heart disease. Assays for measuring homocysteine levels in biological fluids are known in the art. For example, specific assays are disclosed by Tan et al., U.S. Pat. No. 5,998,191 (1999), the contents of which are incorporated herein by reference.

Techniques for calculating risk of cardiovascular disease from a plurality of factors are known in the art. One example is the “Cardiovascular Risk Manager” of D. Cuypers (U.S. Pat. No. 5,396,886 (1995)), the contents of which are incorporated herein by reference. Additional examples are provided by the American Heart Association in Anderson et al. (Circulation 83: 356-362 (1991)) and the World Health Organization (Erica Research Group, The Second European Heart Journal 12: 291-297 (1991)).

Both male and female individuals may be analyzed for risk of cardiovascular disease by the presence or absence of the TaqIB polymorphism. Due to the small number of coronary heart disease events in the group of female individuals in the Framingham Offspring Study, a statistically significant correlation of the association of cardiovascular disease with the absence of the TaqIB polymorphism were made in male individuals only. However, the findings made in this study are also applicable to female individuals.

Detection of the TaqIB polymorphism is accomplished by examination of both copies of the CETP gene in an individual. The TaqIB polymorphism is characterized by the absence of a TaqI restriction endonuclease site in the first intron of the CETP gene. One reliable detection method is to isolate genomic nucleic acid from the individual and examine relevant sequences of the first intron of the CETP gene. The relevant sequences may be isolated by PCR amplification of a suitable section of the first intron of the CETP gene. These sequences can be analyzed by restriction analysis of the fragment for the presence or absence of a TaqI restriction site at the position which corresponds to nucleotide 277 of the first intron of the gene. A suitable section of the first intron is characterized as containing nucleotide 277 and sufficient surrounding nucleotides, such that if the relevant TaqI site were present, the resulting amplified nucleotide would serve as substrate for cleavage. Preferably, the suitable section is between 100 and 1000 base pairs in length, with the putative restriction site located in a central, asymmetrical position within the section, such that cleavage at that site generates two bands which are easily and accurately discernable from each other, and from an undigested band when size fractionated (e.g., on a DNA gel).

In a preferred embodiment, the suitable section of the first intron is 535 base pairs in length. This section may be amplified using the forward primer 5′-CACTAGCCCAGAGAGAGGAGTGCC -3′ and the reverse primer 5′-CTGAGCCCAGCCGCACACTAAC -3′. It is within the abilities of one of skill in the art to devise additional primers which will amplify sections of the nucleic acid suitable for use in the present invention.

The presence of the sequence unique to the TaqIB polymorphism can alternatively be identified, or ruled out, by other methods common in the art. One such method is direct sequencing of the relevant nucleotides. Another method is probing the relevant nucleic acid sequences with labeled oligonucleotide probes which specifically hybridize to one or the other allele, followed by detection of the label to identify allele presence. These and additional methods of detection of a polymorphism are commonly known in the art and within the ability of one of average skill, and as such the present invention encompasses their use.

The mechanism by which the TaqIB polymorphism affects CETP activity is not known. Without wishing to be bound by theory, it is unlikely that the nucleotide sequence change at the location of the TaqI site represents a functional mutation. The most plausible explanation is that the polymorphism is in linkage disequilibrium with a still unknown functional mutation in the CETP gene. Once this functional mutation is identified, the B1 and B2 alleles can alternatively be determined by identification or absence of the functional mutation.

Another aspect of the present invention relates to the use of the TaqIB polymorphism as a marker for decreased atherogenic lipid profile in an individual. The presence of the TaqIB polymorphism correlates with decreased HDL-C levels in men and women, and also for decreased apoA-I levels in men. Statistically relevant correlations of the TaqIB polymorphism with decreased HDL-C levels and decreased apoA-I levels in the individuals of the study are detailed in the Exemplification section below. These results indicate that the CETP gene locus plays a significant role in determining HDL-C variability, apoA-I levels, and LDL size. These associations translate into a less atherogenic lipid profile in individuals of both genders which possess the TaqIB polymorphism. Identification of the TaqIB polymorphism in an individual by the above described methods can therefore also be applied to determining risk for decreased HDL-C levels and for decreased apoA-I levels, to ascertain risk of developing other such pathologies which result from or correlate with such decreases.

Another aspect of the present invention relates to a diagnostic kit for determining susceptibility to the development of cardiovascular disease in an individual. The kit comprises components required for the performance of the above indicated methods for assessing risk for the development of cardiovascular disease in an individual. This includes, without limitation, components for the identification of the TaqIB polymorphism in an individual. Preferably, the components allow the discernment between heterozygosity and homozygosity in the individual. In one embodiment, the kit comprises oligonucleotide primers for the amplification of a suitable section of the first intron of the CETP gene encompassing the TaqI restriction site of the TaqIB polymorphism of the CETP gene, specific examples of which are described above. In another embodiment, the kit comprises alternate means for identifying the TaqIB polymorphism. Other components for the PCR and restriction digestion analysis may optionally be included in the kit. Preferably, the kit of the present invention also contains components for assessment (referred to herein as indicators) of other known factors in cardiovascular disease development. Such factors are also discussed in detail above. The form of the indicators will depend on the factors which are assessed, and can be determined by a practitioner of average skill in the art.

### Exemplification

Subject Characteristics

To investigate the frequency and phenotypic association of the TaqIB CETP polymorphism at the population level, a total of 2876 subjects (1411 males and 1505 females) who participated in the Framingham Offspring Study, and who had lipid values available off lipid altering medication, were analyzed. Table 1 provides a summary of the demographic, genotypic and biochemical characteristics of the participants according to gender. The mean age of men and women at examination was 51.6 and 51.2 years, respectively. Although a similar proportion of men and women were smokers (23.4% and 22.8%, respectively), male subjects smoked more cigarettes per day (5.8±12.5) than the female subjects (4.7±10.3; p<0.016), and over half of the female participants (54.2%) were post-menopausal. There was no significant difference in the frequency of the B2 allele between men and women and the distribution of alleles was consistent with Hardy-Weinberg equilibrium. Alcohol consumption, body mass index (BMI), plasma LDL-C, total apoB, triglyceride and glucose levels were significantly higher in men compared to women, and total HDL-C, HDL₂-C and HDL₃-C concentrations were significantly higher in female participants. The ApoE genotype distribution was similar in men and women (P=0.398).

TABLE 1
Demographic, Genotypic, and Biochemical
Characteristics of Framingham Offspring Study Participants
According to Sex

| | | | P |
|---|---|---|---|
| | Men | Women | (Men vs |
| | (n = 1411) | (n = 1505) | Women) |
| TaqIB-CETP genotype | | | |
| B1B1, % | 428 ± 30.3 | 477 ± 31.7 | — |
| B1B2, % | 713 ± 50.6 | 754 ± 50.1 | — |
| B2B2, % | 270 ± 19.1 | 274 ± 18.2 | — |
| B2 allele frequency | 0.444 | 0.433 | — |
| ApoE alleles | | | |
| E2, % | 12.0 | 14.7 | — |
| E3, % | 67.2 | 62.9 | — |
| E4, % | 20.8 | 22.4 | — |
| Age, y | 51.6 ± 10.1 | 51.2 ± 9.7 | 0.247 |
| BMI, kg/m² | 27.6 ± 3.9 | 25.9 ± 5.3 | <0.001 |
| TC, mmol/L | 5.28 ± 0.96 | 5.30 ± 1.01 | 0.394 |
| LDL-C, mmol/L | 3.47 ± 0.85 | 3.28 ± 0.93 | <0.001 |
| HDL-C, mmoL/L | 1.12 ± 0.29 | 1.45 ± 0.39 | <0.001 |
| HDL₂-C, mmol/L | 0.13 ± 0.10 | 0.26 ± 0.15 | <0.001 |
| HDL₃-C, mmol/L | 0.99 ± 0.23 | 1.20 ± 0.28 | <0.001 |
| TG, mmol/L | 1.54 ± 1.12 | 1.23 ± 1.14 | <0.001 |
| ApoA-I, g/L | 1.34 ± 0.24 | 1.55 ± 0.31 | <0.001 |
| ApoB, g/L | 1.02 ± 0.24 | 0.95 ± 0.26 | <0.001 |
| TC/HDL ratio | 5.00 ± 1.50 | 3.90 ± 1.50 | <0.001 |
| Glucose, mmol/L | 5.41 ± 1.48 | 5.03 ± 1.26 | <0.001 |
| Alcohol, oz/wk | 4.0 ± 5.3 | 1.8 ± 2.9 | <0.001 |
| Cigarettes/d (in smokers) | 5.8 ± 12.5 | 4.7 ± 10.3 | 0.016 |
| Postmenopausal, % | — | 54.2 | — |
| On estrogen therapy,* % | — | 12.9 | — |

Values are mean ± SD or percentages: TC indicates total cholesterol; TG, triglycerides.
*Includes hormonal replacement therapy and the use of oral contraceptives.

Association of the TaqIB Polymorphism with Variations in Plasma Levels of Lipids, Lipoproteins, Apolipoproteins and CETP Activity

In men and women, the three genotype groups were equivalent with respect to age and BMI, as indicated in Table 2. Male homozygotes for the B1 allele had lower HDL-C levels (1.07″0.27 mmol/L) as compared with B1B2 (1.14″0.28 mmol/L) and B2B2 subjects (1.18″0.34 mmol/L); p<0.001. Likewise, female homozygotes for the B1 allele had lower HDL-C levels (1.40″ 0.38 mmol/L) as compared with B1B2 (1.46″ 0.39 mmol/L) and B2B2 subjects (1.53″ 0.40 mmol/L); p<0.001. Similar associations were noted for apoA-I values. The higher HDL-C levels associated with the B2 allele were due to increases in both HDL₂-C and HDL₃-C subfractions. A significant association was noted between the TaqIB genotype and CETP activity. Both men and women carriers of the B2 allele had significantly lower CETP activity than those homozygotes for the B1 allele. In both genders, there were no statistically significant differences among the genotype groups in the plasma levels of total cholesterol, LDL-C and apoB. These results were confirmed by the variance component approach and revealed that TaqIB accounts for about 1% of the variability in HDL-C.

TABLE 2
Plasma Levels of Lipids, Lipoproteins, and Apollpoproteins
of Framingham Offspring Study Subjects
According to TaqIB-CETP Genotypes

| | B1B1 | B1B2 | B2B2 | P* | P† |
|---|---|---|---|---|---|
| Men | | | | | |
| n | 428 | 713 | 270 | | |
| Age, y | 51.2 ± 10.3 | 52 ± 10.0 | 51.3 ± 10.1 | 0.313 | |
| BMI, kg/m² | 27.9 ± 4.0 | 27.50 ± 3.80 | 27.6 ± 3.8 | 0.169 | |
| TC. mmol/L | 5.28 ± 0.93 | 5.25 ± 0.96 | 5.22 ± 0.96 | 0.639 | 0.889 |
| LDL-C, mmol/L | 3.49 ± 0.83 | 3.47 ± 0.88 | 3.41 ± 0.85 | 0.288 | 0.363 |
| HDL-C, mmol/L | 1.07 ± 0.27 | 1.14 ± 0.28‡ | 1.18 ± 0.34§ | <0.001 | <0.001 |
| HDL₂-C, mmol/L | 0.12 ± 0.09 | 0.14 ± 0.10 | 0.15 ± 0.11§ | <0.001 | 0.033 |
| HDL₃-C, mmol/L | 0.95 ± 0.21 | 1.00 ± 0.22‡ | 1.03 ± 0.26§ | <0.001 | <0.001 |
| TG, mmol/L | 1.63 ± 1.16 | 1.52 ± 1.14 | 1.45 ± 0.95 | 0.059 | 0.098 |
| ApoA-I, g/L | 1.32 ± 0.25 | 1.35 ± 0.23 | 1.37 ± 0.24§ | 0.017 | 0.025 |
| ApoB, g/L | 1.03 ± 0.25 | 1.02 ± 0.24 | 1.00 ± 0.25 | 0.135 | 0.662 |
| HDL-C/ApoA-I | 0.81 ± 0.14 | 0.84 ± 0.13 | 0.86 ± 0.13 | <0.001 | <0.001 |
| TC/HDL, ratio | 5.3 ± 1.5 | 4.9 ± 1.5‡ | 4.8 ± 1.6§ | <0.001 | 0.011 |
| CETP, nmol · L^{-f} · h⁻¹ | 160 ± 10.0 | 156 ± 10.0 | 139 ± 9.0 | 0.026 | 0.045 |
| VLDL size, nm | 49.12 ± 10.24 | 48.52 ± 9.23 | 47.34 ± 8.58 | 0.054 | 0.649 |
| LDL size, nm | 20.56 ± 0.60 | 20.69 ± 0.58‡ | 20.80 ± 0.53§ | <0.001 | <0.001 |
| HDL size, nm | 8.83 ± 0.37 | 8.92 ± 0.40‡ | 8.98 ± 0.45§ | <0.001 | <0.001 |
| Women | | | | | |
| n | 477 | 754 | 274 | | |
| Age, y | 51.2 ± 9.7 | 50.8 ± 9.41 | 51.3 ± 10.1 | 0.413 | |
| BMI, kg/m² | 25.6 ± 5.4 | 25.8 ± 5.12 | 26.5 ± 5.5 | 0.081 | |
| TC, mmol/L | 5.28 ± 0.98 | 5.30 ± 1.03 | 5.33 ± 1.03 | 0.901 | 0.794 |
| LOL-C, mmol/L | 3.34 ± 0.93 | 3.28 ± 0.91 | 3.23 ± 0.98 | 0.297 | 0.383 |
| HDL-C, mmol/L | 1.40 ± 0.38 | 1.46 ± 0.39‡ | 1.53 ± 0.40§∥ | <0.001 | <0.001 |
| HDL₂-C, mmol/L | 0.24 ± 0.15 | 0.26 ± 0.14 | 0.28 ± 0.17§∥ | 0.008 | <0.001 |
| HDL₃-C, mmol/L | 1.16 ± 0.28 | 1.20 ± 0.29 | 1.25 ± 0.29§∥ | <0.001 | <0.001 |
| TG, mmol/L | 1.21 ± 0.86 | 1.24 ± 1.38 | 1.23 ± 0.84 | 0.834 | 0.646 |
| ApoA-I, g/L | 1.52 ± 0.28 | 1.55 ± 0.32 | 1.57 ± 0.32 | 0.040 | 0.097 |
| ApoB, g/L | 0.95 ± 0.24 | 0.94 ± 0.27 | 0.95 ± 0.28 | 0.775 | 0.648 |
| HDL-C/ApoA-I | 0.92 ± 0.15 | 0.94 ± 0.16 | 0.97 ± 0.15 | 0.003 | <0.001 |
| TC/HDL ratio | 4.0 ± 1.5 | 3.9 ± 1.50 | 3.7 ± 1.30§∥ | 0.006 | <0.001 |
| CETP, nmol · L⁻¹ · h⁻¹ | 178 ± 11.0 | 159 ± 10.0‡ | 148 ± 11.00§∥ | <0.001 | <0.001 |
| VLDL size, nm | 43.99 ± 8.59 | 44.11 ± 8.40 | 45.81 ± 8.89§∥ | 0.019 | 0.129 |
| LDL size, nm | 21.05 ± 0.52 | 21.07 ± 0.46 | 21.09 ± 0.41 | 0.547 | 0 194 |
| HDL size, nm | 9.35 ± 0.45 | 9.40 ± 0.43‡ | 9.44 ± 0.46§ | 0.027 | <0.001 |

Values are mean ± SD.
*After adjustment for the familial relations.
†After adjustment for the familial relalions, age, BMI, smoking, alcohol intake, use of β-blockers, menopausal status and estrogen therapy (in women), and Apoe.
Shown at significant differences bctween the B1B1 and B1B2 (‡), B1B1 and B2B2 (§), and B1B2 and B2B2 (∥) groups after adjustment for the familial relationships, age, BMI, smoking, alcohol intake, use of β-blockers, menopausal status and estrogen therapy (in women) and Apoe.

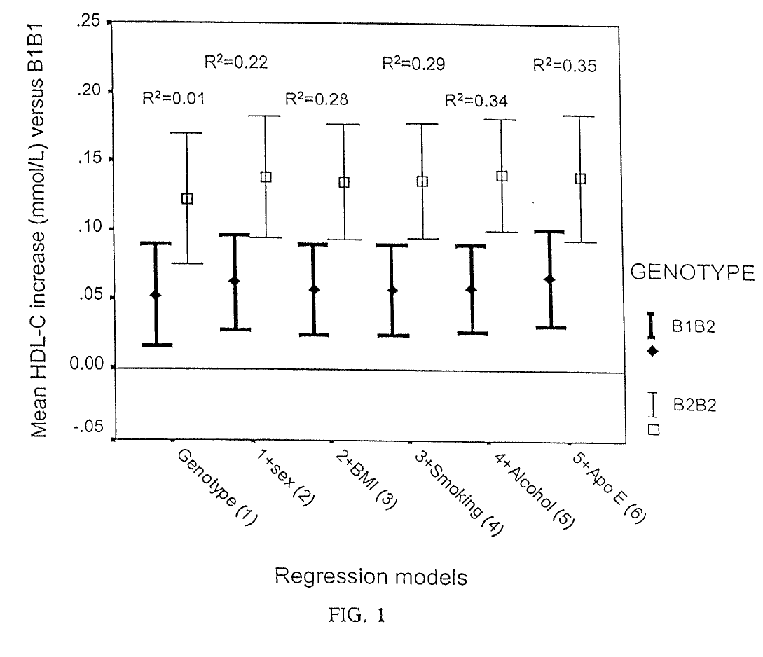

To test the consistency of the association between the CETP TaqIB genotype and HDL-C levels, a sensitivity linear regression analysis was carried out as described below under the heading of Methods of the Invention. FIG. 1 shows regression coefficients and 95% confidence intervals for B1B2 and B2B2 genotypes, respectively, as compared with B1B1 when each indicated variable was included into the linear regression models (Models 1 to 6). First, the only variables included were dummies for TaqIB genotype (Model 1). This genetic factor accounted for 1% of the variability of HDL-C (p<0.001). The initial regression coefficients for B1B2 and B2B2, after controlling for the gender effect (Model 2), were 0.06 (95% CI: 0.03-0.09) mmol/L; p<0.001, and 0.14 (95% CI: 0.09-0.18) mmol/L, respectively; p<0.001. When other variables were progressively added to the core model: BMI, tobacco smoking, alcohol consumption and apoE genotypes, only slight variation of the initially estimated values for the regression coefficients were observed, revealing an independent association of the TaqIB polymorphism with HDL-C levels with a strong consistency, whatever additional environmental or genetic factor was considered. The final model explained 35% of the variability of HDL-C in the population, and the regression coefficient for B1B2 and B2B2 were 0.07 (95% CI: 0.03-0.10) mmol/L and 0.14 (95% CI: 0.09-0.18) mmol/l, respectively (p<0.001).

To gain better understanding of the metabolic basis of the association of higher HDL-C levels with the B2 allele in men and women, lipoprotein subclass profiles were measured using automated NMR spectroscopy. From these measurements, it was determined that this association was specifically due to a significant increase in the large HDL subfraction (8.8-13.0 nm). In males, the HDL-C concentrations (mmol/L) in this HDL subfraction were 0.31±0.27, 0.37±0.29, and 0.45±0.37 for B1B1, B1B2, and B2B2 subjects, respectively (p<0.001). No changes were observed for the small and intermediate size HDL subfractions. These data were consistent with an increase in HDL size in male carriers of the B2 allele as demonstrated by NMR (8.83±0.37; 8.92±0.40 and 8.98±0.45 nm for B1B1, B1B2 and B2B2 subjects, respectively; p<0.001) as well as by an increase in the HDL-C/ApoAI values (indicated in Table 2). In addition to the genotype associations seen with the HDL subfractions, a significant association between this polymorphism and LDL subfractions was observed in men. The B2 allele was associated with increased levels of the large LDL subtraction (1.77±0.89 and 1.94±0.88 mmol/L for B1B2 and B2B2, respectively) as compared with B1B1 subjects (1.64±0.86 mmol/L). Conversely, B1B1 men had increased levels of the small LDL fraction (0.86±0.65 mmol/L) as compared with B1B2 (0.79±0.60 mmol/L) and B2B2 (0.80±0.65 mmol/L) (p=0.031). Therefore, the B2 allele was associated with increased particle size for both HDL and LDL after adjustment for familial relationships, age, BMI, smoking, alcohol intake, use of beta-blockers, and ApoE genotype. In women, a similar effect was noted with the large HDL subfraction. The concentrations were 0.76±0.43, 0.81±0.42, and 0.87±0.44 for B1B1, B1B2, and B2B2 female subjects, respectively (p<0.001). The associations between the B2 allele and LDL size observed in men were not detected in women. Consequently, a genotype/HDL particle size association similar to that shown for men was demonstrated for women after adjustment for the variables indicated above, as well as for menopausal status and estrogen therapy. However, no genotype differences were observed for LDL size.

CETP TaqIB Genotype and Risk of Coronary Heart Disease

To examine the associations of the TaqIB polymorphism with coronary heart disease (CHD) risk, subjects on lipid lowering medications were also included in the analysis. In this analysis, CHD was present in 163 men and 62 women. When CHD prevalence in men was examined at exam 5 with respect to the absence (B1B1) or presence of the B2 allele (B1B2 or B2B2) by chi square analysis, a significantly (p=0.035) lower frequency of carriers of the B2 allele (58.7% vs. 70.6%) among those subjects with positive CHD was demonstrated. Likewise, the odds ratio for CHD associated with the presence of the B2 allele was 0.696 (95% CI: 0.50-0.98; p=0.035). After adjusting for age, BMI, systolic blood pressure, diabetes, smoking, and alcohol consumption, the odds ratio remained at 0.700 (95% CI: 0.46-1.05), but the statistical significance dropped to p=0.090. After additional adjustment for the previous factors plus beta blockers use, cholesterol-lowering drugs, TC and HDL-C, the odds ratio was 0.735 (95% CI: 0.46-1.162; p=0.188). These odds ratios were similar after excluding those subjects on lipid-lowering medications. There were too few CHD cases in the women of the study to draw definitive conclusions about the association between the TaqIB polymorphism and CHD risk in women. No significant association between the presence of the B2 allele and CHD risk was found by chi square analysis (75.8% vs 67.9%, p=N.S.) or by logistic analysis in the women.

Methods of the Invention

Subjects

The details of the design and methods of the Framingham Offspring Study have been presented elsewhere (Feinleib et al., Prev. Med. 4: 518-525 (1975)). Starting in 1971, a total of 5124 subjects were enrolled (Kannel et al., Am. J. Epidemiol. 110: 281-290 (1979)). Blood samples for DNA were collected between 1987 and 1991. Lipid phenotypes, DNA, and information on CHD risk factors were available for 1411 men and 1505 women who attended the 4^{th} and 5^{th} examination visits of the Framingham Offspring Study conducted between 1987 and 1995, and who had lipid values available off lipid-altering medication. Nearly all subjects were Caucasians. Data on smoking, blood pressure, height, weight, and diabetes were obtained on these subjects as previously described (Kannel et al., Am. J. Epidemiol. 110: 281-290 (1979); Dawber et al., Am. J. Public Health 41: 279-286 (1951)). CHD included the presence of myocardial infarction, angina pectoris, coronary insufficiency and coronary death. All suspected CHD events were reviewed by a panel of three physicians to ascertain the presence of CHD. Subjects taking a lipid-lowering medication (n=100) were included for the analyses of CHD prevalence at exam 5, but excluded in all other analyses.

Plasma Lipid, Lipoprotein, Apolipoprotein and CETP Measurements

Twelve-hour fasting venous blood samples were collected in tubes containing 0.1% EDTA. Plasma was separated from blood cells by centrifugation and immediately used for the measurement of lipids. Plasma total cholesterol (TC), HDL-C and triglyceride levels were measured as previously described (Cupples et al., Circulation 85: 111-118 (1992)). HDL-C was measured after precipitation of ApoB-containing lipoproteins with dextran-magnesium sulfate (Warnick et al., Clin. Chem. 28: 1379-88 (1982)). Low density lipoprotein-cholesterol (LDL-C) concentrations were estimated with the equation of Friedewald et al. (Clin. Chem. 18: 499-502 (1972)). Coefficients of variation for total cholesterol, HDL-C, triglyceride measurements were each less than 5 percent (McNamara and Schaefer, Clin. Chim. Acta. 166: 1-9 (1987)). Plasma levels of apolipoprotein (apo) AI and apoB were measured by non-competitive enzyme-linked immunosorbent assay (ELISA), using affinity-purified polyclonal antibodies (Schaefer and Ordovas, Metabolism of the apolipoproteins A-I, A-II, and A-IV. In: Segrest J, Albers J, editors. Methods in Enzymology, Plasma Lipoproteins, Part B: Characterization, Cell Biology and Metabolism. Academic Press, 1986: 420-442); Ordovas et al., J. Lipid Res. 28: 1216 (1987)).

Plasma lipoprotein concentrations and subclasses distributions were determined by proton nuclear magnetic resonance (NMR) spectroscopy as previously described (Otvos et al., Clin. Chem. 38: 1632-1638 (1992); Otvos, J. D., Measurement of lipoprotein subclass profile by nuclear magnetic resonance. In: Rifai N, Warnick G R, Dominiczak M H, editors. Handbook of lipoprotein testing. Washington: AACC Press, 1997: 497-508). Each profile displays the concentrations of six very low density lipoproteins (VLDL), one intermediate density lipoproteins (IDL), three LDL, and five HDL subclasses and the weighted-average particle sizes of VLDL, LDL and HDL. The 10 lipoprotein subclass categories used were the following: large VLDL and remnants (80-220 nm), intermediate VLDL (35-80 nm), small VLDL (27-35 nm), large LDL (21.3-27.0 nm), intermediate LDL (19.8-21.2), small LDL (18.3-19.7 nm), large HDL (8.8-13.0 nm), intermediate HDL (7.8-8.8 nm), and small HDL (7.3-7.7 nm). Levels of VLDL subclasses are expressed in units of triglyceride (mmol/L), and those of LDL and HDL subclasses in units of cholesterol (mmol/L). LDL and HDL subclass distributions determined by gradient gel electrophoresis and NMR have been shown to be closely correlated (Otvos et al., Clin. Chem. 38: 1632-1638 (1992)). However, it should be noted that given the characteristics of this methodology, there could be some overlap between the IDL fraction and the small VLDL, as well as with the large LDL subfraction. Nevertheless, this should not have a major effect over the associations examined given the low concentrations of IDL found in fasting plasma of normal subjects.

CETP activity was determined using a CETP Activity Kit by Roar Biomedical, Inc. (New York, N.Y.). This kit includes a donor (synthetic phospholipid and cholesteryl ester particles) and acceptor particles (VLDL). The fluorescent neutral lipid is present in a self-quenched state when contained within the core of the donor. The CETP mediated transfer is determined by the increase in fluorescence intensity as the fluorescent neutral lipid is removed from the self quenched donor to the acceptor. Briefly, for each sample assayed, 10 ul of plasma was diluted (1:10) in 90 ul of sample buffer (10 mM tris, 150 mM NaCl, 2 mM EDTA, pH 7.4). In a fluorescent compatible microtiter plate (Dynex Laboratories), 20 ul of the plasma dilution was combined with 4 ul of donor and 4 ul of acceptor in a total volume of 200 ul, and incubated for 3 hours at 37° C. The assay was read in a fluorescent spectrometer at excitation wavelength of 465 nm and emission wavelength of 535 nm. A standard curve was used, according to manufacturer guidelines, to derive the relationship between fluorescence intensity and mass transfer. Plasma controls were run in each plate to account for plate to plate variation. For standardization, the unquenched fluorescence intensity of the fluorescent cholesteryl ester contained within the donor particle core was determined by dispersing 5 ul of donor (fluorescent CE concentration 146 ug/ml—reported by manufacturer) in 2 ml of 100% isopropanol. Serial dilutions of the dispersion were made to generate a standard curve of fluorescence intensity (ex. 465 nm/em. 535 nm) vs. mass of fluorescent CE. The fluorescence intensity transferred in the assay of plasma samples was applied to the standard curve to determine mass transfer. The intra- and interassay coefficients of variation were less than 3%.

DNA Analysis

Genomic DNA was isolated from peripheral blood leucocytes by standard methods (Miller et al., Nucleic Acids Res. 16: 1215 (1989)). CETP genotype was performed as described by Fumeron et al. (J. Clin. Invest. 96: 1664-1671 (1995)). A fragment of 535 base pairs in intron 1 of CETP gene was amplified by polymerase chain reaction (PCR) in a DNA Thermal Cycler (PTC-100, MJ Research, Inc., Watertown, Mass.), using oligonucleotide primers (Forward: 5′-CACTAGCCCAGAGAGAGGAGTGCC-3′ SEQ ID NO: 1 and Reverse: 5′-CTGAGCCCAGCCGCACACTAAC-3′ SEQ ID NO: 2). Each amplification was performed using 100 ng of genomic DNA in a volume of 50 μL containing 40 pmol of each oligonucleotide, 0.2 mM dNTPs, 1.5 mM MgCl₂, 10 mM Tris, pH 8.4 and 0.25 U of Taq polymerase. DNA templates were denatured at 95° C. for 3 min and then each PCR reaction was subjected to 30 cycles with a temperature cycle consisting of 95° C. for 30 sec, 60° C. for 30 sec, and 72° C. for 45 sec, and finally an extension at 72° C. for 5 min. The PCR products were subjected to restriction enzyme analysis by digestion with 4 units of the restriction endonuclease TaqI for 16 μL of PCR sample at 65° C. for 2 h in the buffer recommended by the manufacturer (Gibco-BRL) and the fragments separated by electrophoresis on an 1.5% agarose gel. After electrophoresis, the gel was treated with ethidium bromide for 20 minutes and DNA fragments were visualized by UV illumination. The resulting fragments were 174 bp and 361 bp for the B1 allele, and 535 bp for the uncut B2 allele. ApoE genotype was carried out as previously described (Hixson and Vernier, J. Lipid Res. 31: 545-548 (1990)).

Statistical Analyses

To compare men and women who participated in the study, chi-square tests for categorical measures and two-sample t tests for continuous measures were employed. The allele frequency of the B2 allele and APOE alleles was estimated with the chromosome counting method and use of a chi-square test to compare the frequency in men and women. To evaluate the relationship between the CETP genotypes and lipid levels, analysis of covariance (ANCOVA) techniques which accounted for the familial relationships among the members of the study (mostly siblings and cousins) were used. Two approaches were used to accomplish these analyses. First, a repeated measures approach was employed, which assumed an exchangeable correlation structure among all members of a family, (PROC MIXED, SAS). Since this approach does not accurately represent the true correlation structure within these pedigrees, a measured genotype approach (Boerwinkle and Utermann, Am. J. Hum. Genet. 42: 104-112 (1988)) as implemented in SOLAR, a variance component analysis computer package for quantitative traits measured in pedigrees of arbitrary size (Almasy and Blangero, Am. J. Hum. Genet. 62: 1198-1211 (1998)), was also employed. The latter approach fully accounts for the different types of relationships within a pedigree in performing an analysis of variance on the defined genotypes. In these analyses, several different models were used to adjust for potential confounders. First, essentially crude results were obtained, which accounted only for the family structure; second, adjustments were made for age, body mass index (BMI), smoking, alcohol consumption, beta-blockers, and (in women) menopausal status and hormonal replacement therapy. In the final analysis, ApoE genotypes were added to the model with E2/E2 and E2/E3 in one group, E3/E4 and E4/E4 in a second group, and E3/E3 as the reference group. Subjects with E2/E4 genotypes, of which there were very few, were excluded.

A sensitivity analysis was carried out to estimate the validity and precision of the regression coefficients for the CETP genotypic variables when additional independent terms were included into the model. Because similar results were obtained for both sexes, data from men and women were analyzed together to improve statistical power. Regression coefficients and 95% confidence intervals for B1B2 and B2B2 genotypes as compared with B1B1 were calculated by fitting several linear regression models with dummy variables for categorical and interaction terms as follows: model 1: CETP genotype (B1B1, B1B2 and B2B2). Model 2: model 1+gender. Model 3: model 2 +BMI. Model 4: model 3+tobacco smoking (non smoker and smoker). Model 5: model 4+alcohol consumption (consumption and no consumption). Model 6: model 5+apoE genotypes (E2, E3 and E4). In all cases, the first category was taken as reference. Regression diagnostics were employed to check the assumptions and to assess the accuracy of computations.

Finally, using a chi-square analysis, the odds of prevalent CHD at exam 5 for those with the B1B2 or B2B2 genotypes relative to those with the B1B1 genotype were estimated. CHD includes myocardial infarction, angina pectoris, and coronary insufficiency. To adjust the estimated odds ratio for covariates, logistic regression was employed. Generalized estimating equations with a logit link was also applied to account for the correlation among the observations, and obtained essentially the same results. Hence, the results are reported assuming independent observations.

Relevant statistical analyses are presented below.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. A method for assessing risk for the development of cardiovascular disease in an individual, comprising:
a) isolating nucleic acid from the individual;
b) analyzing the nucleic acid for the presence of the TaqIB polymorphism of the cholesteryl ester transfer protein gene;
c) determining from the analysis of step b) whether the individual:
i) is homozygous for the TaqIB polymorphism;
ii) is heterozygous for the TaqIB polymorphism; or
iii) does not possess the TaqIB polymorphism; and
d) assessing the risk for the development of cardiovascular disease in the individual on the basis of determinations made in step c).

2. The method of claim 1 wherein a determination in step c) that the individual does not possess the TaqIB polymorphism correlates with high increased risk for the development of cardiovascular disease.

3. The method of claim 1 wherein a determination in step c) that the individual is heterozygous for the TaqIB polymorphism correlates with moderate increased risk for the development of cardiovascular disease.

4. The method of claim 1 wherein a determination in step c) that the individual is homozygous for the TaqIB polymorphism correlates with no increased risk for the development of cardiovascular disease.

5. The method of claim 1 wherein the susceptibility is assessed on the basis of the determinations made in step c) in combination with additional determinations of one or more known factors of cardiovascular disease risk.

6. The method of claim 5 wherein the factor is genetic.

7. The method of claim 5 wherein the factor is environmental.

8. The method of claim 7 wherein the environmental factor is dietary.

9. The method of claim 1 wherein the individual is male.

10. The method of claim 1 wherein the individual is female.

11. The method of claim 1 wherein the nucleic acid is genomic DNA.

12. The method of claim 11 wherein the nucleic acid is analyzed for the presence of the TaqIB polymorphism by PCR amplification of a suitable section of the first intron of the cholesteryl ester transfer protein gene followed by restriction analysis of the fragment for the presence of a TaqI restriction site at a position corresponding to nucleotide 277 of the first intron, wherein the presence of the TaqI restriction site indicates the absence of the TaqIB polymorphism, and the absence of the TaqI restriction site indicates the presence of the TaqIB polymorphism.

13. The method of claim 12 wherein the suitable section of the first intron is 535 base pairs in length and is amplified using the forward primer 5′-CACTAGCCCAGAGAGAGGAGTGCC-3′ and the reverse primer 5′-CTGAGCCCAGCCGCACACTAAC-3′.

14. The method of claim 1 wherein the cardiovascular disease is selected from the group consisting of myocardial infarction, angina pectoris, coronary insufficiency and coronary death.

15. A kit for assessing risk for the development of cardiovascular disease in an individual, comprising oligonucleotide primers for the amplification of a suitable section of the first intron of the cholesteryl ester transfer protein gene encompassing the TaqI restriction site of the B1 allele of the CETP gene, the presence of the TaqI restriction site being indicative of the absence of the TaqIB polymorphism.

16. The kit of claim 15 wherein the oligonucleotide primers are the forward primer 5′-CACTAGCCCAGAGAGAGGAGTGCC-3′ and the reverse primer 5′-CTGAGCCCAGCCGCACACTAAC-3′.

17. The kit of claim 15 which further includes indicators for additional known factors of cardiovascular disease risk.
